(19) United States
(12) Patent Application Publication
(10) Pub. No.: US 2005/0250155 A1
(43) Pub. Date: Nov. 10, 2005

(54) Multiple marker characterization of single cells

(75) Inventors: Stephen A. Lesko, Baltimore, MD (US); Paul O.P. Ts'o, Ellicott City, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Cell Works Diagnostics, Inc., Abingdon, MD (US)

(21) Appl. No.: 11/182,897

(22) Filed: Jul. 18, 2005

Related U.S. Application Data
(63) Continuation of application No. 09/430,175, filed on Oct. 29, 1999.
(60) Provisional application No. 60/106,118, filed on Oct. 29, 1998.

(51) Int. Cl. C12Q 1/68; G01N 33/574; G01N 1/30; G01N 33/48

(52) U.S. Cl. 435/6; 435/40.5; 435/7.23

(57) ABSTRACT

Methods use concurrent multiple cellular probes conjugated to fluorescent compounds of different wavelengths to characterize single cells that have been isolated from a body fluid using density gradient centrifugation. Specific antibodies, peptides, nucleotides or oligonucleotides are used as probes for both identification and characterization of a single cell.

### TECHNICAL FIELD OF THE INVENTION

The present invention concerns the characterization of multiple cellular markers on a single cell via the concurrent use of multiple fluorescent probes.

### BACKGROUND OF THE INVENTION

Characterizing and monitoring a single cell environment, and more particularly an abnormal cell, such as a foreign cell or cell modified from its healthy mode such as a cancer cell or a virally-infected cell, involves concurrent testing of multiple markers on a single cell using fluorescent probes.

When molecules absorb light they subsequently dispose of their increased energy by various means, one of which is the emission of light of longer wavelengths. When a molecule is irradiated with visible or ultraviolet light, it may undergo an electronic transition during which the molecule absorbs a quantum of energy, and an electron is excited from the orbital it occupies in the ground state to another orbital of higher energy. The ultraviolet and visible spectra recorded for molecules are absorption spectra. Most excited states are short-lived and the major fate of the absorbed energy in the ultraviolet region is reemission of light as phosphorescence or fluorescence. When the emission is of short duration, such as 10⁻⁸ to 10⁻⁹ seconds for return of the excited molecules to the ground state, the process is called fluorescence. Fluorescence occurs when molecules absorb light in internal molecular transfers wherein light is remitted at a longer wavelength. The fluorescent properties of antibody molecules and other organic dyes that can be attached to them provide the basis for a number of analytic methods, one of which is immunofluorescence (Bright, Analytical Chem., 60:1031, (1988); Guilbault (Ed) In: Practical Fluorescence, Second Ed., Marcel Dekker (1990); McGowan et al., J. Histochemistry & Cytochemistry, 36(7):757-762, (1988); Jones et al., Biochemical & Biophysical Research Communications, 167(2):464-470 (1990).

Fluorescent antibody techniques involve a variety of methods including direct fluorescent, indirect fluorescent, mixed antiglobulin, and sandwich techniques. The direct fluorescent staining reaction involves a process, wherein the fluorescent-labeled probe, such as antibody, is specific for the molecule (e.g., antigen) of interest. Another direct technique involves a “sandwich” reaction used to identify antibody rather than antigen in tissue samples. Antigen is added to tissue and is bound by specific antibody present in the cell. Specific fluorescein-labeled antibody to antigen is added and reacts with the antigen, which is now fixed to the antibody in the cell.

Indirect fluorescent staining reactions may involve a multiple-step process, wherein step one of a simple reaction concerns an unlabeled antibody (i.e., primary antibody) that is specific for an antigen, and other steps may concern a fluorescent-labeled antibody of another species (e.g., secondary or tertiary antibody such as goat anti-rabbit immunoglobulin) that binds to the unlabeled antibody. Another indirect method involves a mixed antiglobulin reaction, wherein antigens present on the primary antibody are used to react to binding sites on the secondary antibody. The immunoglobulin antigens are present on the cell and the anti-immunoglobulin antibody is used to bind labeled immunoglobulin to the cell surface immunoglobulin.

The indirect fluorescent technique is known for it's increased sensitivity due to the first or primary antibody providing more binding sites for the secondary antibody than was provided by the tissue antigen. Although increased sensitivity is associated with indirect fluorescent methods, the number of markers that can be tested per cell is limited. One reason is a spatial limitation due to the increased number of secondary and tertiary antibody consuming more of the cellular surface per antigen to be characterized.

The major disadvantage of the indirect fluorescent method is the limited availability of monoclonal antibodies of different species. In general, monoclonal antibodies are generated in mice, rats, goats, rabbits, and sheep. So there is a limited number of species to use. It is difficult to differentiate between two probes when, for example primary antibodies raised in mice because the secondary antibody, such as goat anti-mouse, would recognize both probes. Thus, a serious limitation is caused because a different species is needed for each primary antibody probe.

A comparison of direct and indirect fluorescent antibody techniques illustrates the spatial limitations caused by steric hindrance when using the indirect methods. The direct fluorescent techniques deal directly with specific fluorescent-labeled antibody binding to an antigen and allows the maximum number of markers to be tested. Although the primary antibody provides more binding sites for second antibody in the indirect methods than was provided by tissue antigen and increases the sensitivity of the technique, critical cellular surface space is blocked and prevents the optimum number of immunological surface markers from being tested. (Stewart Sell, “Antigen-Antibody Reactions,” In: Basic Immunology, Elsevier Publisher, New York, p. 137, (1987)).

Kuebler discusses staging of circulating cancer cells (U.S. Pat. No. 5,529,903). Concentrates of circulating cancer cells in a leukapheresis white blood cell fraction are assayed using PCR and subsequent culture in order to identify oncogenic markers. Kuebler does not address the characterization of single cells by concurrently using multiple probes linked to fluorescent labels.

Flow cytometry is another method for detecting the presence of cancer cells in the blood of patients. Using flow cytometry with multiple immunofluorescent markers, there is good correlation between tumor cell number, chemotherapy and clinical status in blood (Racila et al., Proc. Natl. Acad. Sci., 95:4589-4594, (1998)). This technique has provided prognostic information about the cancer cells in the patient's blood (Racila supra), bone marrow (Gross et al., Proc. Natl. Acad. Sci., 92:537-541, (1995)0 and apheresis products (Simpson et al., Exp. Hematol., 23:1062-1068, (1995)).

There is a growing list of cellular markers available for evaluating cells, especially immune cells, foreign or diseased cells, such as cancer cells. Although the first tumor marker was identified in 1847, the usefulness of tumor markers only was recognized in the 1960s in gastrointestinal cancer.

A number of groups have successfully developed methods of separating breast cancer cells from blood and/or bone marrow using anti-cytokeratin monoclonal antibodies to epithelial antigens of the cancer cells. Epithelial cells are not normally present in these samples unless they are from cancer spread. (Martin et al., Exp. Hematol., 26:252-264, (1998); Berios, supra; Naume et al., J. Hematother., 6:103-114, (1997)). Heatly et al., J. Clin. Pathol., 48:26-32, (1995) carried out a study of cytokeratin expression in benign and malignant breast epithelium to examine changes in cytokeratin profile. An antibody in their study, CAM 5.2, is specific for cytokeratins and was positive for the majority of adenocarcinomas as well as fibroadenoma and fibrocystic disease.

Invasive potential has been linked with cell proliferation markers MiB1/Ki67 and proliferating cell nuclear antigen (PCNA). Using these two types of cell growth markers, Kirkegaard (Anat. Pathol., 109:69-74, (1997)) found that proliferation of astrocytomas, as measured by image cytometry of MiB1/Ki67 and PCNA, correlated significantly with histologic grade and patient survival.

MiB1/Ki67, introduced by Gerdes (Int. J. Cancer, 31:13-20 (1983)), provides a direct means of evaluating the growth fraction of tumors in histopathology and cytopathology (Key et al., Lab Invest., 68;629-636, (1993)0. Sasano (Anticancer Res., 17:3685-3690, (1997)) found a significant correlation between cell proliferation marked by MiB1/Ki67 expression with invasive ductal carcinoma. Vielh (Am. J. Clin. Pathol., 94:681-686, (1990) conducted a study of immunohistologic staining (Ki67 index) versus flow cytometry using Ki67 monoclonal antibody. Proliferative indices were deemed to be better using immunohistochemical techniques than flow cytometry.

PCNA is also a good marker of cell proliferation, with evidence of deregulated expression in some neoplasms and occasional upregulation in benign tissue (El-Habashi et al., Acta. Cytol., 41:636-648, (1997); Hall et al., J. Pathol., 162:285-294, (1990); Leong and Milios, Appl. Immunohistochem., 1:127-135, (1993); Matthews et al., Nature, 309:374-376, (1984); Siitonen et al., Am. J. Pathol., 142:1081-1088, (1993); Galand and Degraef, Cell Tissue Kinet., 22:383-392, (1989)).

Staging, including the determination of aggressiveness of the cancer in biopsy material using markers of cell growth, cell growth inhibition, aneuploidy or hormone receptor status is possible. Currently, there is a need to detect metastatic potential in circulating cancer cells in “at risk” patients. Properly staging cancer aids in the selection of appropriate therapeutic interventions based upon this information, and allows one to monitor the status of the patient, i.e., prognosis, drug treatment, and any possible remissions or disease progressions. The disclosed inventions provide improved methods to detect, enumerate, and provide information concerning circulating cancer cells and have the potential to revolutionize the diagnosis and treatment of cancer. Such methods are useful to provide an evaluation of a patient's disease status, to determine appropriate treatment intervention, and to monitor the effectiveness of such intervention.

Staging, including the determination of aggressiveness, of the cancer in biopsy material has relied on a mixture of probes, such as probes directed to cell growth, cell growth inhibition, aneuploidy, or hormonal receptor status. These data derived from biopsy studies have shown good correlation to patient outcome factors. However, there has been no research into applying the concurrent measurement of multiple probes directed to cellular markers on or in single cells, especially cancer and/or immune cells, and most especially circulating cancer cells isolated from blood samples of patients.

The concurrent multiple characterization of a single circulating isolated from a body fluid, such as a cancer cell, provides a health assessment and/or a cancer characterization profile of the mammal, depending upon the selection of markers. In particular, the isolation and characterization of a small number of circulating cancer cells in a body fluid sample from a mammal provides an opportunity to assess the number and nature of each cancer cell type. Concurrent multiple characterization is especially important when only 1 or 2 circulating cancer cells are isolated from each sample, when a small volume of blood is processed or the donor has very few circulating cancer cells for examination. Thus, there is a need to use multiple markers concurrently to characterize these few cells, i.e., 1 to 20, isolated circulating cancer cells maximally in order to assess the nature of the cancer. Further, since circulating cancer cells usually comprise heterogeneous population of cells, there is a need to characterize each type of cancer cell that is isolated from the circulation of a mammal. Thus, characterizing each cell within the scope of the present invention provides more information about each sample to be tested. The ability to characterize a small number of heterogeneous cancer cells based on the presence or absence of multiple characteristics on each cell isolated from a mammal's circulation may provide information useful for staging and evaluating treatment options.

### SUMMARY OF THE INVENTION

In accordance with the instant invention, methods for characterizing a single cell comprising multiple somatic and genetic expression of cellular markers in a single cell environment, wherein probes directed to said cellular markers have the ability to fluoresce.

An object of the invention is a method of establishing a characterization profile comprising a method of characterizing a single cell environment, wherein the concurrent measurement of multiple cellular markers using fluorescent probes, wherein said probes emit different wavelengths of light to distinguish multiple cellular markers expressed in a single cell using fluorescent microscopy. Preferably, a method of establishing a characterization profile involves repeated testing of a subject to accumulate data over varying time periods.

An object of the instant invention relates to a method of characterizing a single cell preparation comprising adherence of a cell preparation onto a surface, fixing said cell preparation with a fixative solution, incubating such a cell preparation containing fixed cells with multiple probes directed to desired cellular markers, wherein said multiple probes have the ability to fluoresce, (which are excitable at different wavelengths), and examining the cells by fluorescence microscopy for identification of cells positive for each selected cellular marker. A preferred object of the invention is to characterize circulating cancer cells that are isolated using a negative selection protocol through density gradient centrifugation process, and more preferably, a double density gradient centrifugation process.

Another object of the invention is a method to characterize a single cell environment from a mammal in order to establish a multiple marker characterization profile of said mammal. One preferred object of the invention is a method to characterize single cells from an individual with a disease, such as an individual with cancer or an individual suspected of having cancer to provide a multiple characterization profile of the cancer.

Another object of the invention is to characterize the cellular markers of a single cell environment using probes conjugated to fluorescent compounds, wherein fluorescent dyes or compounds are selected to allow one to distinguish between the markers by elimination of overlapping wavelengths of the light being emitted by each fluorescently-labeled probe using a fluorescent microscope with appropriate spectral filters, wherein each probe may be imaged with no major interference.

### BRIEF DESCRIPTION OF THE DRAWINGS

FIG. 1 illustrates the concurrent measurement of various markers using fluorescent probes.

### DETAILED DESCRIPTION OF THE INVENTION

The instant invention relates to methods of characterizing a single cell environment comprising detection of a variety of cellular markers concurrently via fluorescent probes as observed by a fluorescence microscopy. Preferably a probe, which is directed to a cellular marker, is conjugated to a fluorescent compound to form a probe-fluorophore conjugate that can be detected selectively via a microscope with an appropriate fluorescent filter or filters, such as an optical filter set.

### Multiple Marker Characterization

The invention is directed to the use of multiple fluorescent probes that bind to cellular markers, wherein fluorescent dyes of the probes do not interfere with the ability to distinguish one marker from the next marker of the particular group of cellular markers and probes of interest for characterization. In a preferred embodiment of the invention, a probe may be either a biological probe, which is a protein or peptide, and more preferably an antibody or a molecular probe, which may be a DNA or RNA molecule. Preferably, the selection of fluorescent probes for testing multiple cellular markers comprises probes conjugated to different fluorescent compounds that when excited are able to emit light of specific wavelengths. Concurrent testing of cellular markers via multiple probe-fluorophore conjugates within a single cell environment provides a profile of the characteristics of a cell or a group of cells. In a preferred embodiment of the invention, fluorescent probes are selected from a group consisting of a mixture of fluorescent probes that emit wavelengths of light between 400 nanometers and 850 nanometers and with the use of filters of appropriate band width and wavelength, one can distinguish between said markers by elimination of overlapping wavelengths of light being emitted by each fluorescent-labeled probe; such optical filter sets that are capable of detection of the specific emission spectra for each probe. More preferably, the fluorescent probes emit light with wavelengths between 430 namometers to 510 nanometers, 482 namometers to 562 nanometers, 552 namometers to 582 nanometers, 577 namometers to 657 nanometers, 637 namometers to 697 nanometers, 679 namometers to 763 nanometers, and 745 namometers to 845 nanometers, and most preferably, the fluorescent probes emit light with peak wavelengths of about 470 nanometers, 522 nanometers, 567 nanometers, 617 nanometers, 667 nanometers, 721 nanometers, and 795 nanometers .

“Concurrent” shall mean that the presence or absence of markers for a single cell environment as tested at the same time. A “single cellular environment” shall mean a single cell or a group of cells isolated from one source, such as a blood sample or a cultured cell sample derived from a mammal, such as a human. Such a group of single cells may be heterogeneous. The number of cells isolated from a body fluid sample may vary depending upon the source of cells. For example, the variation of cells isolated from a small volume of blood, e.g., 20 ml blood sample, to a larger volume of blood, e.g., leukapheresis sample, may vary from 1 to 250 cells (although some samples may have zero cells isolated from a particular sample). However, most 20 ml blood samples have only a few cells isolated for characterization, generally 1 to 20 cells, and more generally 1 to 5. Thus, characterization of a single cell environment is maximized using a variety of cellular markers on a limited number of cells using multiple marker characterization methods of the present invention. This can generate valuable information about the cell of interest at that point in time.

A “cellular marker” shall mean any somatic or genetic marker of a cell that is detectable and/or measurable. A cell may be determined to be positive or negative for any selected cellular marker providing that there is a corresponding probe that binds to the marker. Further, quantifying and/or measuring the intensity of each marker of interest is a preferred embodiment of the invention. Biological and molecular characterization may involve characterizing single cancer cells based on antibody binding activity to an antigen (e.g., receptor, intracellular protein and/or peptide) to measure proliferative and motility activities, for example. Further, immunological profiling may provide information concerning the binding capability of the cell and/or the motility of the cell regarding metastatic potential. Specifically, cancer cell antigens may be targeted either alone or in combination with molecular markers including, but not limited to, epidermal growth factor receptor, epithelial membrane antigen, epithelial specific antigen, estradiol, estrogen receptor, tumor necrosis factor receptor superfamily (e.g, tumor necrosis factor (TNF) and Fas), ferritin, follicle stimulating hormone, actin, gastrin, hepatitis B core antigen, hepatitis B surface antigen, heat shock proteins, Ki-67, lactoferrin, lamin B1, lutenizing hormone, tyrosine kinases, MAP kinase, microtubule associated proteins, c-Myc, myelin basic protein, myoglobulin, p16, cyclin-dependent kinases (e.g., P27,p21), p53, proliferation associated nuclear antigen, pancreatic polypeptide, viral proteins (e.g., papillomavirus, cytomegalovirus, hepatitis, etc.) proliferating cell nuclear antigen, placental lactogen, pneumocystis carinii, progesterone receptor, prolactin, prostatic acid phosphatase, prostate specific antigen, pS2, retinoblastoma gene product, S-100 protein, small cell lung cancer antigen, serotonin, somatostatin, substance P, synaptophysin, oncogene, tumor associated probes, including AFP, β₂ microglobulin, CA 19-9 antigen, CA 125 antigen, CA 15-3 antigen, CEA, cathepsin, cathepsin D, p300 tumor-related antigen (e.g, such as detected by an M344 monoclonal antibody), collagen, melanoma, prostate specific antigen, HER-2/neu (e.g., p185, which is a protein product of HER-2/neu oncogene), and apoptotic genes and/or proteins (e.g., Bcl-2). Some of the probes may be more relevant to some cancers than others. For example, a positive identification of CA 125 may indicated longer patient survival (Scambia, et al., Eur. J. Cancer, 32A(2):259-63). Likewise, CA 15.3 antigen may be more important to squamous cell carcinoma antigen (SCC) with respect to predicting a chemotherapeutic response in cervical patients (Scambia, supra).

In general, characterization methods of the present invention would include any antibody of choice, e.g., a probe that reacts to an antigen, e.g., a cellular marker, of choice. For example, specific cells can be identified using various probes to specific cell types, such as lymphocytes (e.g., T lymphocytes, B lymphocytes, and natural killer cells, macrophages, dendritic cells, langerhan cells, etc.). Any antibody directed to a specific cell type may be used within the scope of the invention. In particular, CD2 and/or CD3 may be used to identify a T lymphocyte, CD14 may be used to identify a macrophage, and CD19 may be used to identify a B lymphocyte. Other antibodies that may be used are well known in the literature. Examples of suitable leukocyte antibodies include CD2, CD3, CD4, CD5, CD7, CD8, CD11a, CD11b, CD11c, CD14, CD15, CD16, CD19, CD20, CD28, CD34, CD36, CD42a, CD43, CD44, CD, 45, CD45R, CD45RA, CD45RB, CD45RO, CD57, CD61, and the like. Antibodies targeted to human CD45, CD3, CD19, CD14, and CD36 are preferable. For example, a CD45 antibody is useful for recognizing a CD45 leukocyte common antigen (LCA) family, which is comprised of at least four isoforms of membrane glycoproteins (220, 205, 190, and 180 kD). In particular, the use of the negative separation for enriching circulating epithelial cells can be purified with a mixture of anti-human antibodies, such as CD45, CD 14, and CD3. Antibodies are commercially available (Transduction Laboratories Ltd., UK; Southern Biotechnology Associates, GA, and PharMingen, CA). In addition to monoclonal antibodies, antibodies may comprise polyclonal antibodies, Fab fragments, and/or peptides. DAPI, Hoechst, propidium iodide are counterstains that are useful for staining DNA in the nucleus of a cell and acridine orange is useful for staining RNA.

In one embodiment of the invention, a characterization protocol may include combination staining (e.g., fluorescence staining) and fluorescent in situ hybridization (FISH) (FISH protocol and probes can be found, for example, in Meyne et al., in Methods of Molecular Biology, 33:63-74 (1994)). For example, specific nucleic acid sequences are suitable as probes for cancer cells. In particular, molecular probe design may include, but is not limited to, chromosomal centromere probes such as those for Chromosome 18, 5′-Cy3-TT-Cy3-TT-Cy3 -GAG ATG TGTGTACTCACACTAAGA GAATTGAACCACCGTTTTGAAGGAGC-3′; Chromosome 17, 5′-CY5-TT-CY5-TT-CY5-TGT TTC AAA CGT GAA CTT TGA AAG GAA AGT TCA ACT CGG GGA TTT GAA TG-3′; Chromosome 7, 5′-CY5-TT-CY5-TT-CY5-GCT GTG GCA TTT TCA GGT GGA GAT TTC AAG CGA TTT GAG GAC AAT TGC AG-3′; and mRNA Probe Design such as Cytokeratin 14 mRNA probe, 5′-CY3-TT-CY3-TT-CY3-GGA TTT GGC GGC TGG AGG AGG TCA CAT CTC TGG ATG ACT GCG ATC CAG AG-3′; Cytokeratin 19 mRNA Probe, 5′-CY3-TT-CY3-TT-CY3-ATC TTG GCG AGA TCG GTG CCC GGA GCG GAA TCC ACC TCC ACA CTG ACC TG-3′; MUC I (EPISIALIN) mRNA Probe, 5′-FITC-TT-FITC-TT-FITC-TTG AACTGTGTCTCCACGTCGTGGAC ATTGA TGGT AC C TTCTCGG AAG GC-3′; and Estrogen-mRNA probe, 5′-CY5-TT-CY5-TT-CY5-GTG CAG ACC GTG TCC CCG CAG GGC AGA AGG CTG CTC AGA AAC CGG CGG GCC AC-3, and in particularly, probes for the centromere regions of chromosome 7 (e.g., CGATTTGAGG ACAATTGCAG), chromosome 18 (e.g., GTACTCACAC TAAGAGAATT GAACCACCGT), chromosome X (e.g., GACGATGGAGTTTAACTCAGG, TCGTTGGAAACGGG AATAA TTCCCATAACTAAACACAAACA, AAGCCTTTTCCTTTATCTTCACAGAAAGA) may be targeted. A sequence length of about 20 to about 60 nucleotides can be used, preferably a length of about 40-45. Cancer cells can also be identified by polymerase chain reaction (PCR) techniques, which techniques and probes are well known to those in the art.

A cellular marker shall mean any somatic or genetic marker of a cell that is detectable and/or measurable. A cell may be determined to be positive or negative for any selected cellular marker. Further, quantifying and/or measuring the intensity of each marker of interest is a preferred embodiment of the invention. In a preferred embodiment of the present invention, isolating and characterizing cells isolated from a mammal with cancer, suspected of having cancer, or at risk for developing cancer, such as a human, is a means of establishing a customized characterization profile for each sample in order to determine the presence or absence of cancer, and to stage the progression, recurrence, or remission of the cancer. The relevance of this embodiment is captured in the following scenario. The presence of circulating breast origin cells in a blood sample may indicate that epithelial cells are sloughed off into the blood and that a characterization profile showing low growth factors, high growth inhibitor factor, diploid status, normal DNA content, and an estrogen receptor positive would indicate that these cells are not cancerous. However, if these isolated epithelial cells were characterized as being aneuploid and as having high growth potential, for example, the assessment of the patient would be very different. Preferably, each cell to be characterized can be tested to determine relevant markers for that particular cell type. For example, a cancer cell may be characterized using a mixture of probes directed to particular cellular markers in order to identify the origin of the cell (e.g., prostate), the specific type of cell (e.g., epithelial), non-specific molecular markers (e.g, p53), and unique or more cell specific in nature (e.g., hormones, such as estrogen, progesterone, androgen; Her-2/neu). Aneuploidy means any deviation from an exact multiple of the haploid number of chromosomes, and in the present invention refers to hyperploidy (such as, triploid, tetraploid, ect.) in the context of a cancer cell. The molecular characterization of single circulating cancer cells of the present invention, which may be continuously evolving in their neoplastic progression, may provide valuable information concerning the staging and/or the aggressiveness of the cancer. Epidermal growth factor (EGF) is overexpressed in breast and ovarian cancers. The overactivity of the EGF receptor has been linked to one third of all epithelial cancers, such as breast, bladder, lung, kidney, head and neck, and prostate. The HER2/neu receptor is elevated or mutated in cancer patients in comparison to cancer-free individuals. Breast cancer patients that produce the HER-2 protein in excessive quantities have a poor prognosis. Clinical studies using antibodies against the HER-2 receptor are underway in breast cancer patients. The goal is to block the HER-2 oncogene receptor with antibodies.

The term “multiple” shall mean 4 or more cellular markers and/or probes for characterizing a single cell environment. A preferred embodiment of the invention is that about 5 or more, about 6 or more, or 7 markers and/or probes can be tested per single cell environment. Seven probes can be tested concurrently with the proviso that each positive marker can be identified for each selected fluorescent-labeled probe in the multiple probe-fluorophore conjugate set to be used for characterization of the cell using a microscope that contains a large number of filter sets corresponding to the different emission wavelengths. Preferably, 4 or more fluorescent-labeled probes per slide containing cells isolated from a body fluid using density gradient centrifugation can be tested concurrently with the proviso that each positive marker can be identified for each selected fluorescent-labeled probe in the multiple probe-fluorophore conjugate set to be studied per slide or per isolated sample containing the cell; and more preferably, 5, 6, or 7 fluorescent-labeled probes per slide containing the single cell environment can be used for multiple marker characterization. It is noted that mercury lamp is used for fluorescent probes that emit light within wavelengths in the range of 450 to 725 nanometers.

The source of the cells for multiple cellular characterization comprises any cell-containing fluid, preferably a body fluid, such as a natural body fluid or an enriched body fluid, tumor samples, or cultured cells isolated from a body fluid or tumor, and more preferably an enriched cell sample containing cancer cells, and most preferably, isolated circulating cancer cells in blood, urine, or bone marrow obtained via density gradient centrifugation (U.S. Pat. No. 5,962,237). An “enriched body fluid” comprises a leukapheresis or apheresis fraction, and the like.

Cells for characterization may include, but not be limited to, any cell derived from a mammal or cultured in vitro, the following normal and abnormal cell types: epithelial, endothelial, skeletal, bone, bone marrow cells, circulating cells derived from body fluids or body tissues, nerve, and muscle. An abnormal cell type shall mean a cell that deviates from its normal mode of somatic and/or genetic expression, such as a diseased cell, such as a cancer cell, a virally-infected cell, or a cell involved in graft-versus-host disease. Most preferably, cells are circulating cancer cells that comprise many different cancers, including, but not limited to, epithelial cancers such as prostate, breast, liver, kidney, colon, rectum, gastric, esophageal, bladder, brain, ovary, pancreas, and lung. Other cancers in the form of a sarcoma, (e.g., a fibrosarcoma or rhabdosarcoma), a hematopoietic tumor of lymphoid or myeloid lineage, or another tumor, including, but not limited to, a melanoma, teratocarcinoma, neuroblastoma, or glioma. The evaluation of the characteristics of a circulating cancer cell or a group of circulating cancer cells isolated from a mammal, such as a human, may provide a current assessment of the health of the source of the cells.

The development of a characterization profile of the present invention has a useful application for clinically monitoring the number and type of normal and abnormal cells. A preferred embodiment of the invention involves measuring the number and characteristics of circulating epithelial cancer cells isolated from a body fluid sample, such as breast, prostate, kidney, etc., isolated from samples of body fluids for monitoring the disease progression, if any. More particularly, the invention relates to a health assessment of a mammal at a particular point in time. The development of a characteristic profile of isolated circulating cancer cells is valuable to determine metastatic potential, to monitor for cancer recurrence, and to assess therapeutic efficacy. Breast cancer serves as one example of the importance of establishing a multiple characterization profile. About 30 to 50% of breast cancer patients will develop metastatic breast cancer, which kills the patient. The earlier a patient is aware of metastatic cancer cells (i.e., cells identified with high growth potential and aneuploidy, for example), the greater chance of receiving earlier drug intervention and hopefully, a greater chance of survival. Currently, a blind period may exist from the time of diagnosis until metastatic cancer develops. This period varies from patient to patient and becomes a critical period to monitor all breast cancer patients. The concurrent measurement of multiple markers for characterizing intact circulating cancer cells is valuable since the number of isolated cells many vary from 1 to over 250 cells per sample. Of course, processing a patient sample that establishes that no circulating cancer cell is present is valuable information. Repeat testing is recommended to confirm any negative test data. Patient monitoring is highly recommended to establish that the cancer continues to remain localized, is in remission, or that the patient is cured. Thus, determining the presence or absence of circulating cells is in itself an important step to establish for each patient, and furthermore to establish repeatedly for each patient. A series of repeated negative tests may be followed by the development of positive isolation of circulating cancer cells, which then may be characterized within the scope of the invention. This new information establishes evidence that cancer still exists in the body and is established sufficiently in the body to produce cancer growth capable of generating cancer cells in the circulation. Many times the actual secondary source of the cancer in the body is unknown.

A prognostic or therapeutic review may include probes, such as antibodies, peptides, nucleotides or oligonucleotides, which provide cell identification, growth, growth inhibition (e.g., cell resting state), ploidy state, and hormonal receptor assessment. For example, CAM 5.2 is an antibody, which reacts with cytokeratins and is useful to identify an epithelial cancer cell. Anti-P27 is a probe to evaluate a cell's resting or quiescent state. Anti-MiB1/Ki67 and PCNA are two probes to evaluate cell growth potential. Hormone receptor or gene status is helpful for determining the value of a therapeutic or a combination of interventions, including multiple drug treatment or a radiation in combination with drug therapy, or prognostic information. Ploidy state is important for prognostic information concerning the identification of cancer or an inheritable disease. For example, evaluation of chromosomes 1, 17, and/or 18 can be determined using probes (some of which are listed above, for example).

Expression of various cellular markers can possibly correlate with each other. For example, an inverse relationship between PCNA-MiB1/Ki67 and P27 expression may exist. Estrogen receptor negative cells, which are most aggressive, may correlate directly with MiB1/Ki67 and PCNA expression, and may have an inverse correlation with P27 expression. Polyploid cells, which are considered aggressive, may have high MiB1/Ki67 and PCNA expression, and may be low in P27 expression. One particular probe-fluorophore conjugate set envisioned for the instant invention includes probes labeled with fluorescent compounds (e.g., probe-fluorescent dye) such as MiB1-CY3, PCNA-CY3.5 or TEXAS RED™, P27-CY5, Cytokeratin-FITC, PSA-AMCA, or the DNA counterstain (DAPI) per sample or per slide.

A preferred embodiment of this multiple marker test takes advantage of the state-of-the-art computerized fluorescence microscopy to provide an invaluable tool to assess: (1) whether there are cancer cells circulating in the bloodstream (2) whether these cells have the potential to divide within the bloodstream or to anchor and form a metastatic secondary tumor site. Optionally, optimization of the test involves identifying a set of markers on a slide containing cells from cultured cell lines for characterization. One set of probe-fluorophore conjugates directed a set of cellular markers could be applied and the slide read on the microscope, then the coverslip removed and another set of probe-fluorophore conjugates could be applied; allowing many markers to be tested on a single sample. The XY coordinate memory feature of the microscope could be used to relocate the cells of interest if required due to multiple staining sessions. The multiple focal-plane Z axis merge feature of the microscope allows visualization and enumeration of chromosome number when the chromosomes are located at different planes within the cell. A number of cell lines would be tested to ensure that the test is reproducible and sensitive for all types of cancer cells.

Some of these markers will correlate with patient outcome. A combination of isolated cancer cells from the blood of patients who are at risk for metastatic breast cancer and subsequent staining for expression of cytokeratin, P27, MiB1/Ki67 and/or PCNA, presence of estrogen receptor and ploidy of chromosomes 1, 17, and 18 should provide some statistical correlation between these markers and the prognostic factors of the patient. Patients at risk for breast cancer metastases are likely to have cytokeratin positive breast cancer cells in the blood circulation. It is expected, in patients that have cells with metastatic potential in their blood to have high growth markers (MiB1/Ki67 and PCNA), and low expression of growth-inhibition marker P27. If a patient is a responder to an estrogen receptor drug, such as tamoxifen, it is expected that upon treatment over time, the circulating cancer cells isolated from blood will decrease in number with decreased expression of MiB1/Ki67 or PCNA, and will continue to be estrogen receptor positive. In particular, these specific techniques can be used to find, identify and characterize breast cancer cells that are possibly forming micro-metastases in the blood or secondary sites. It is expected that markers for metastasis or aggressiveness, such as aneuploidy, and estrogen receptor negativity will have a direct correlation to markers of cell growth (MiB1 and PCNA) and inverse correlation to cell arrest markers (P27). The long-term goal is that this information will be helpful to the patient in multiple ways, such as early detection and elimination of lymph node dissection, prognostic information, and indication of whether the type of cancer would respond to hormone therapy, and indication for therapy appropriateness, and for examining blood replacement products.

To visualize multiple markers within the same cancer cell in order to provide a characterization profile for an individual patient may include, but is not limited to, an evaluation of the aggressive potential of the circulating cells. The circulating cells could simply be innocent travelers in the bloodstream due to cell death within the primary tissue site, or aggressive killer cancer cells circulating like warriors looking for a place to take hold. This innovative approach to patient care can be conducted before tumors are detected by current scanning methods. This technique can also be used to monitor effectiveness of therapy and used to change the course of therapy if necessary.

To visualize multiple markers within the same cancer cell allows for its characterization and importantly to determine its aggression potential at an early stage. The rationale for this invention is that markers are available that correlate with patient outcome. For example, when a patient has breast cancer, there are often breast origin cells circulating in the blood that may or may not be threatening to the patient. The innovative nature of this research is that an application will be developed to visualize multiple markers on or within the same cell so that, when cells are found, individual cells will be analyzed for hopefully early stage aggression potential.

For example, without being bound to any particular theory, one hypothesis may be that, when circulating breast origin cells are found in circulation they may be cells which have sloughed off from surrounding tissue—not tumor cells. If circulating epithelial cells are isolated then one might expect to find low growth factors, high growth inhibition factor, diploidy and/or estrogen receptor positivity. As the patient's condition worsens, the number of circulating breast cancer cells increases and aggressiveness factors are also expected to increase. Samples of whole blood or aphersis white cell fraction sample mixed with cultured breast cancer cells, or patient samples may be examined for possible interferences that could be present in patient blood, such as lipemic blood or blood that has chemotherapy or hormone therapy drugs.

Application of probe-fluorophore conjugates to circulating cells may indicate malignancy and can provide early warning concerning prognosis or therapeutic success as seen in marker correlation. A rational and systematic approach to choosing markers has been analyzed that could provide prognostic value, either for growth potential (especially non-anchored growth potential or the ability to divide within the blood stream) with subsequent prognostic predictions, or for therapy assessment.

Current literature (and the inventors' experience with multiple markers within a single cell environment) suggests that a choice of markers that could provide prognostic or therapeutic value would include cytokeratins, P27 (cell resting state), MiB1/Ki67 (cell growth) or PCNA (cell growth), estrogen receptor (therapeutic value or prognostic information), and ploidy state (prognostic information) or chromosomes 1, 17, and/or 18. Markers may be found to correlate with each other.

The P27/Kip protein belongs to the recently identified family of proteins called cyclin-dependent kinase inhibitors. These proteins play an important role as negative regulators of cell cycle-dependent kinase activity during progression of the cell cycle. Tsihlias et. Al., Cancer Res., 58:542-548, (1998) found in prostate cancers that increased P27 staining correlates with benign prostatic epithelial components in all tumor sections. Harvat et. Al., (Oncogene, 14:2111-2122, (1997)) reported that exogenous expression of P27 in cultured breast cancer cells induces growth arrest. Assessment of P27 as a prognostic marker in node negative patients has been found to be useful for identifying patients with small, invasive breast carcinoma who might benefit from adjuvant therapy (Tan et. al., Cancer Res., 57:1259-1263, (1997); Katayose et. al., Cancer Res., 57:5441-5445, (1997)). It has been reported that infection of breast cancer cells with recombinant adenovirus expressing human P27 causes high P27 expression in the cells, and a marked decrease in the proportion of cells in the S-phase, or apoptosis (Craig et. al., Oncogene, 14:2283-2289, (1997)). There is an inverse correlation between P27 level and anchorage-independent growth of cancer cells, which could be important in the ability of the cancer cells to metastasize in the blood (Kawada et. al., J. Cancer Res., 89:110-115, (1998)).

Chromosome aneuploidy in breast cancer patients and the relationship to invasiveness in clinical applications have been correlated (Wingren et. al., Br. J. Cancer, 69:546-549, (1994)). Fluorescent In-Situ Hybridization (FISH) can be used, not only to determine overall ploidy, but also to assess the over-representation of under-representation of specific chromosomes in interphase cells. Shackney et. al. (Cytometry, 22:282-291, (1995) found that multiple copies of chromosomes 1, 3 and 17 were accumulated selectively in the cells of individual tumors more frequently then other chromosomes studied. Affiy and Mark (Cancer Genet. Cytogenet., 97:101-105, (1997)) found trisomy of chromosome 8 correlated with stage I and II infiltrating ductal carcinoma of the breast, and other markers that predict aggressive biological behavior.

Breast cancer can be divided into two types according to the estrogen receptor level of the tumor (Zhu et. al., Med. Hypotheses, 49:69-75, (1997)). Estrogen receptor positivity is associated with a 70% response rate to anti-hormonal therapy. In contrast, the response rate is less than 10% among patients whose tumors are estrogen receptor negative. Patients whose tumors are estrogen receptor positive generally achieve superior disease free survival (Rayter, BR. J. Surg., 78:528-535, (1991)).

Correlation of growth factors, inhibitors, estrogen receptors, and aneuploidy have been done in many studies, but not in cancer cells found within the blood circulation. Using flow cytometry, Lee et. al., (Mod. Pathol., 5:61-67, (1992)) found that aneuploidy was significantly related to the loss of estrogen receptors, high histologic grade, high nuclear grade and mitotic rate. Immunohistochemical evaluation of proliferation by staining with anti-Ki67 monoclonal antibody correlated strongly with the mitotic rate. Aneuploid and tetraploid tumors demonstrated higher Ki67 scores than diploid tumors. Correlation was demonstrated between aneuploidy and low levels of estrogen receptor (Fernandes, et. al., Can. J. Surg., 34:349-355, (1991)). Correlation of proliferation markers, estrogen receptors and drug therapy in circulating cells has been done with biopsy material by Makris et. al., (Breast Cancer Res. Treat., 48:11-20, (1998)) in a “first time” study where an early decrease in proliferation marker was shown to relate to subsequent clinical response to tamoxifen therapy. Responders were more likely to be estrogen receptor (ER) positive, with low Ki67. They observed a decrease in Ki67 and ER after 14 days of treatment that was related to subsequent response.

A variety of hormones can be tested, including, but limited to, estrogen, progesterone, androgen, dihydrotestosterone, and testosterone. For example, androgen receptor and androgen receptor gene copy number can be detected in cancer cells isolated from prostate cancer patients. The identification and characterization of circulating prostate cancer cells is especially of interest. Androgens mediate a number of diverse responses through the androgen receptor, a 110 kD ligand-activated nuclear receptor. Androgen receptor expression, which is found in a variety of tissues, changes throughout development, aging, and malignant transformation processes. The androgen receptor can be activated by two ligands, testosterone and dihydrotestosterone, which bind to the androgen receptor with different affinities. This difference in binding affinity results in different levels of activation of the androgen receptor by the two ligands. The androgen receptor acts as a transcriptional modifier of a variety of genes by binding to an androgen response element. The ability to confer androgen specific actions by the androgen response element may depend on other cell-specific transcription factors and cis-acting DNA elements. Testosterone and dihydrotestosterone appear to act upon an identical nuclear receptor. However, in certain instances, they mediate different physiologic responses. For example, dihydrotestosterone, but not testosterone, is capable of mediating full sexual development of the male external genitalia. In some cases, the androgen receptor may induce opposite physiologic responses in similar tissue types depending on their location. For example, in male pattern baldness, activated androgen receptors may suppress the growth of distinct hair follicle populations through initiating stromal-epithelial actions, whereas other hair follicles continue to proliferate. In other cases, altered androgen receptor activity due to its mutation or altered expression may lead to pathology such as recurrence of prostate cancer due to development of androgen independence allowing tumor cell proliferation under androgen deprivation.

Proteins and mRNA levels can be used to test hormonal receptor expression (e.g., androgen and estrogen) and oncogene expression (e.g., p53, HER2, and p21). Tests to characterize hormonal receptor gene copy number and oncogene number detect mutations or single base mutations.

Overexpression of amplified genes is often associated with the acquisition of resistance to cancer therapeutic agents in vitro. A similar molecular mechanism in vivo for hormonal treatment failure in human prostate cancer involves amplification of the androgen receptor (AR) gene. Comparative genomic hybridization shows that amplification of the Xq11-q13 region (the location) is common in tumors recurring during androgen deprivation therapy. High-level androgen receptor amplification is observed in 30% recurrent tumors. Androgen receptor amplification emerges during decreased androgen concentrations (Visakorpi et. al., Nature Genetics, 9:401-406, (1995)).

Determining a response to a drug treatment regimen is another valuable tool to address whether a drug is efficacious by quantifying the number of cells and characterizing the cells for disease progression. In a preferred embodiment of the invention, a baseline characterization profile is established (i.e., the establishment of a first profile) and subsequent characterization profiles would be compared to the baseline. Another application of this invention is to monitor bone marrow or white blood cell transplantation products before entry into a patient.

### Isolation of Circulating Blood Cells

The invention relates to methods of characterizing the single cell environment of any subject comprising evaluating a variety of cell probes conjugated to various fluorescent compounds, wherein such compounds are selected that when excited they are able to emit light of different wavelengths. Preferably, cells isolated from natural and enriched body fluids are characterized. More preferably, circulating cancer cells isolated form blood or blood fractions using density gradient centrifugation are characterized using methods described in U.S. Pat. No. 5,962,237. The selection of substantially pure cancer cells, e.g., 20-80% purity, isolated from the circulation may allow for a more definitive characterization and exploitation of specific methods for using such cells, e.g., staging the cancer, determining drug sensitivity, determining the presence of metastatic cells, and/or developing cancer vaccines. Specifically, the present invention additionally provides methods for isolating circulating cancer cells in natural and enriched body fluids that have been subject to density gradient centrifugation and have been subjected to negative or positive selection to remove all or most white blood cells and/or red blood cells. Generally, isolated circulating cancer cells isolated in natural body fluids are subjected to negative selection to remove as many white blood cells and/or red blood cells as possible and those cancer cells isolated in enriched body fluids, i.e., leukapheresis, are subject to positive selection. In the scope of the present invention, negative selection means a conventional process of binding a non-cancer cell to an antibody, for example, and the bound non-cancer cell is separated from the cancer cells. The negative selection process encompasses both “direct” and “indirect” protocols. For example, a direct negative selection process includes using an antibody bound to a support, e.g., microbead, wherein the antibody binds to a non-cancer cell. Indirect negative selection involves using a “primary” antibody to bind to the non-cancer cell, and a “secondary” antibody, which is bound to a support, to bind to the primary antibody. Circulating cancer cells isolated form non-concentrated body fluids are contaminated with leukocytes. Any binding agent, e.g., antibody, that binds to leukocytes may be used to reduce or eliminate these cells from the cancer cells, e.g., anti-CD45 antibodies or anti-CD3 antibodies. In the scope of the present invention, positive selection means a conventional process of binding a cancer cell by binding agent, such as an antibody, and the bound cancer cell is separated from the non-cancer cells.

Natural body fluids include, but are not limited to, fluids such as blood, enriched blood fractions, saliva, lymph, spinal fluid, semen, amniotic fluid, cavity fluids, and tissue extracts. Various volumes of natural body fluids may be used. Generally, a useful volume of natural body fluid means about 5 to 75 ml of blood is extracted from the patient to be tested. Preferably, about 15 to 25 ml of venous blood, for example, is tested, and most preferably, about 20ml is tested. Twenty milliliters of blood constitutes a ratio of 1:300 to 1:350 of total blood volume.

Naturally enriched or concentrated sources of body fluids include any method of enriching body fluids that contain white blood cells and circulating cancer cells (if present). Preferably, examples of concentrated body fluids include leukapheresis, buffy coat, apheresis and the like (U.S. Pat. No. 5,529,903). Concentrated body fluid samples or fractions, such as apheresis or leukapheresis, are collected by widely available protocols (Technical Manual of the American Association of Blood Banks, Washington, D.C., pp. 17-337, 1981). Generally, a 3-hour period of time is allotted to harvest a concentrated cell fraction containing white blood cells and circulating cancer cells (if present) in 3 liters of blood. Three liters of blood is a significant volume of blood to process for enriched cell fractions that may contain circulating cancer cells since an average human subject contains six to seven liters of blood. The process of capturing these enriched cell fractions allows red blood cells and serum to be re-transfused to the patient. The leukapheresis (U.S. Pat. 5,112,298 and U.S. Pat. No. 5,147,290) and apheresis (U.S. Pat. No. 5,529,903) procedures trap and concentrate cancer cells within a white blood cell (WBC) fraction. Thus, for a long term goal of standardizing molecular and immunological profiling and culturing of cancer cells from individual patients, use of leukapheresis samples may be preferable to support data collected from a natural body fluid because larger number of cells may be isolated from patients.

In contrast to a 20 ml sample of natural body fluid, (e.g., blood), the probability of isolating circulating cancer test in 3 liters of an enriched body fluid becomes increased by as much or greater than 100 to 150 times higher. Characterization data may then become more definitive because more cells are isolated for evaluation. Based upon the derived characterization profiles, critical and beneficial decisions affecting changes in therapeutic treatments may be made for the individual patient providing the leukapheresis sample.

### Conjugation of Fluorophores to Monoclonal Antibodies

Since the antibodies we are using for both identification and characterization of prostate cancer cells are all mouse monoclonals, analyses using more than two antibodies by indirect immunofluorescence are tedious and unworkable. The best way to handle this problem is to directly label each antibody with a different fluorophores. In: Davis, W. C., editor, Monoclonal antibody protocols, (Towata (N.J.): Humana Press; 1995. 215-221, (1995)) a comprehensive survey of procedures and reagents for protein conjugate preparation are provided. The following antibody conjugates using succininmidyl ester derivatives of the fluorophores were prepared: anti-cytokeratin-CY3, anti-Ki67 (MiB1)-FITC, anti-Kip1/P27-Texas Red™, WDZ3 (anti-Prostate Specific Membrane Antigen, which is a mixture of WDZ1 (ATCC #HB-11430) and WDZ2 (ATCC #HB-10494)-Texas Red, anti-P27-CY5, anti-androgen receptor-CY3, anti-Prostate Specific Antigen (PSA)-AMCA and Prostate Specific Acid Phosphatase-Texas Red™, and pepsinogen-Texas Red. The antibodies and fluorescent derivatives are available commercially (e.g., Organon Teknika, Durham, N.C.).

### Characterization of Cells with Labeled Monoclonal Antibodies

Prostate cancer cells are spun onto slides using a cytospin centrifuge (1000 rpm for 10 min.). After air drying for at least two hours, the cells are fixed and permeabilized in 3% paraformaldehyde/1% triton/PBS for four minutes at 4° C. or 2% paraformaldehyde for 10 minutes at 4° C. The cells are then incubated with 3% BSA/PBS or 1% BSA/0.1% Saponin with 4 or 5 labeled antibodies under a coverslip in a humidified chamber. Finally, the slides are washed in PBS at room temperature 2-3 times, 5 minutes each and then mounted in an anti-fade medium containing DAPI for examination by fluorescence microscopy. Images are acquired with a sophisticated microscope (Leica, Germany) equipped with cooled CCD camera and fluorescent filter cubes that can discriminated the 4 to 7 or more, preferably 5 to 6, or more preferably 6 to 7 different fluorescent-labeled antibodies. The images can be merged to produce colored composites to reveal a prostate cell if it stains positive for a prostate-specific antigen-AMCA and cytokeratin-FITC. If the prostate cell has proliferative capacity, it should stain positive for Ki67-CY3 (red) and be positive for Proliferating Cell Nuclear Antigen (PCNA)-Texas Red (if it is in the S-phase of the cell cycle). Non-cycling, quiescent prostate cells should stain positive for P27-Cy5. Appropriate colors can be assigned to CY5 and Texas Red. A recent report (van Oijen, et. Al., Am. J. Clin. Pathol. 110: 24-31, 1998) shows that not all cells containing the Ki67 antigen (MiB1) are actively proliferating cells. This report deals with cells treated with synchronizing inhibitors and cells that overexpress P53 and P21. Because of this recent report, an anti-PCNA antibody is included in the assay to identify proliferating cells in the S-phase of the cell cycle. Identifying these actively dividing cells in the blood of cancer patients should aid in a multi-phasic approach to patient prognosis and treatment.

### Immunodetection of Cells (Cancer Cells)

After completing the fixing step, the liquid is aspirated form the surface of the slide (e.g., a vacuum), and then the labeled probes are added to the sample on the slide in a solvent composed of 100 ml of 1×PBS, 0.5% BSA, 0.1% Saponin, and 0.05% NaN₃. A coverslip is placed onto the sample area. The slide is incubated at room temperature for 60 minutes in a moisture box. The slide is placed in a Coplin jar with 1×PBS at room temperature for 10 minutes. Examples of probe-label conjugates include any mixture of protein or DNA labeled with a fluorescent compound. For example, cytokeratin and WDZ-3 antibody staining involves the preparation of a mixture (30 μl) containing anti-cytokeratin antibody-FITC (CAM 5.2 commercially available from Becton Dickinson) and WDZ-3 antibody-TEXAS RED™ (Cell-Works) at a concentration of 70-150 ng/μl, and preferably at about 100 ng/μl. WDZ-3/TEXAS RED™ conjugate contains a dye/protein ratio of about 2.

### Fluorescent In Situ Hybridization (FISH)

FISH can be used, not only to determine overall ploidy, but also to assess the over-representation or under-representation of specific chromosomes in interphase cells. Other probes may be added to the mixture including chromosome 18 that is labeled to a specific fluorescent compound. For example, aneuploidy of chromosome 18 may be examined using CY3-labeled chromosome 18 on LNCaP prostate cancer cells isolated from blood circulation. Chromosome 18 conjugated to CY3 has a dye/protein ratio of 2. The final concentration of chromosome 18-CY3 is about 70-150 ng/μl, most preferably, about 125 ng/μl.

Preparation of FISH Cocktail:

| FISH Buffer: | | Components | Final Concentration |
|---|---|---|---|
| 280 | μl | 100% Deionized formamide | 28% |
| 200 | μl | 20× SSC | 4× |
| 100 | μl | 10× PBS pH 7.0 | 1× |
| 100 | μl | 10 mg/ml Carrier DNA | 1 μg/μl |
| 50 | μl | 20 mg/ml Carrier tRNA | 1 μg/μl |
| 100 | μl | 50% Dextran sulphate | 5% |
| 150 | μl | 50× Denhardt's* | 7.5× |
| 2 | μl | 500 mM EDTA | 1 mM |
| 18 | μl | Distilled H₂O | |
| 1000 | μl | | |

*50× Denhardt's: 1% polyvinylpyrrolidone, 1% Ficoll, and 2% BSA

FISH Cocktail (Vol./slide): 19.51 μl FISH buffer; 0.5 μl CY3-Chromosome Centromere probe 18 (200 ng/μl). FISH Staining: Add the Fish cocktail onto the sample area on the slide; Place the coverslip on the sample area; Seal the coverslip with rubber cement; Denature the sample at 85° C. for five minutes on a hot plate; Hybridize the sample at 42° C. in oven for four hours in a moisture box; Take off the rubber cement and coverslip form the sample slide very carefully; Wash the slide in a Coplin Jar with 2× Standard Saline Citrate (SSC)/0.1% NP-40 (USB; Cat: 19628) at 52° C. (preheated) for 2 minutes; Air-dry the slide at room temperature; DAPI Counterstain the sample with 14 μl/slide of DAPI in mounting medium (1.0 μg/ml; Vector Lab; Cat. H-12000); Place a coverslip on the sample; Seal the coverslip with FLO-TEXX mounting medium (Lerner Lab; Cat. M770-3); Stand the slide in a dark area at room temperature for at least 10 minutes.

### EXAMPLE 1

Example 1 illustrates the characterization of cancer cells with monoclonal antibodies labeled with fluorescent compounds.

Cytospin preparations were made to test LNCaP cells (a prostate cancer cell line) and white blood cells. Any slide may be used to prepare a cytospin prep. Preferably, a charged slide (VWR Scientific) is used with Shandon Megafunnels/Slide Assembly. The cytospin preparations contain about 5×10⁵ cells/2.5ml. The slides are assembled with megafunnels and are placed in a Shandon Cytopsin-3. Samples are centrifuged at 1,000 rpm with acceleration on high for 10 minutes at room temperature. Open and separate the megafunnel chamber from the slide. Slides may be air dried at room temperature for at least 2 hours and then stored in a slide box until staining. Preferably, slides are air-dried overnight and then fixed at 4° C. in 2% paraformaldehyde for 5 minutes or 3% paraformaldehyde/1% TritonX100 for 4 minutes. For example, Coplin jars are filled with fixative (at about 4° C.), slides are placed in the fixative solution for 10-15 minutes, then slides are rinsed one time with Phosphate Buffered Saline (PBS) and incubated in PBS for 10 minutes. The cells were permeabilized by incubation at RT for 15 minutes with 1.0% Bovine Serum Albumin (BSA)-0.1% saponin in PBS, and then incubated with one or more monoclonal antibodies in the same solution for one hour at room temperature or at 4° C. overnight, (preferably at 4° C. overnight). Next day the cells were washed two times, five minutes each, at room temperature to remove unbound antibody. After mounting in anti-fade medium containing DAPI (Vectashield, Vector Laboratories), cells were examined by fluorescence microscopy, using a microscope (Leica, Germany). Images were acquired with a cooled CCD camera and appropriate fluorescent filter cubes.

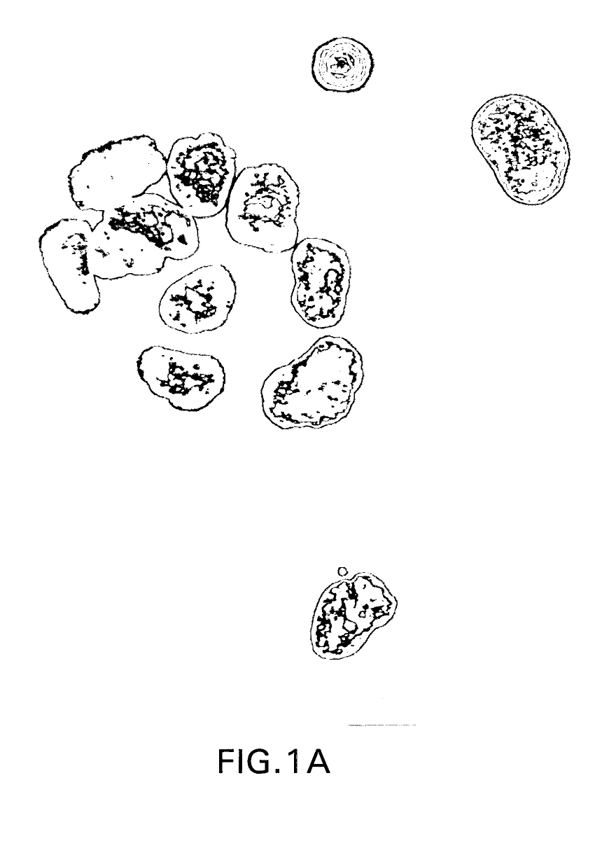

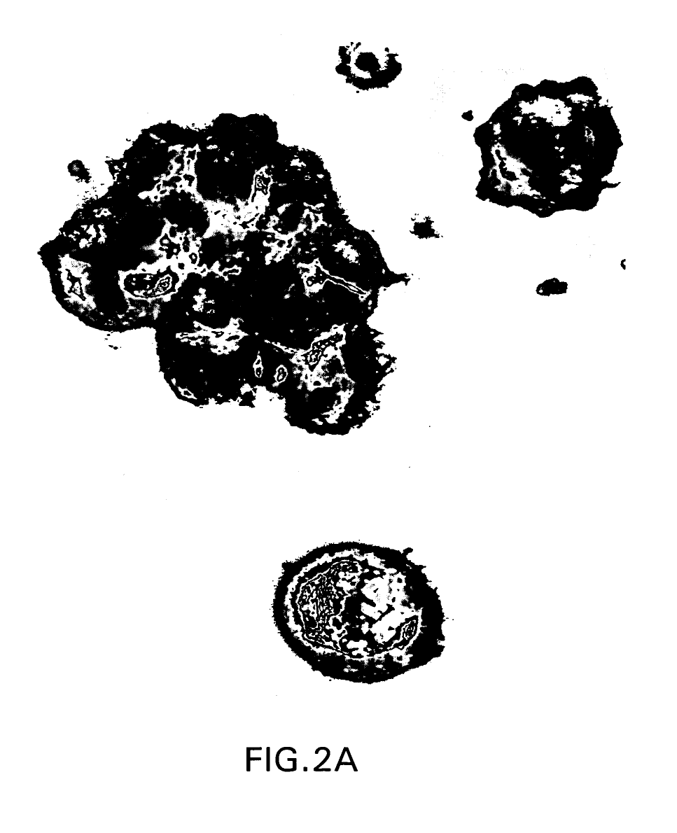

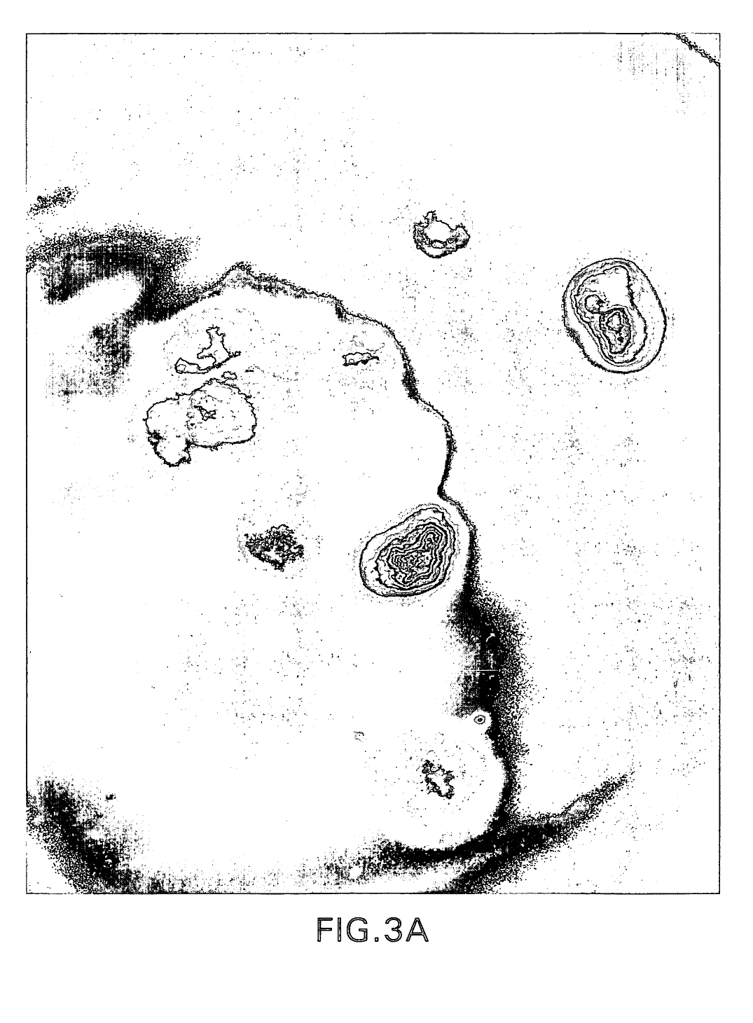

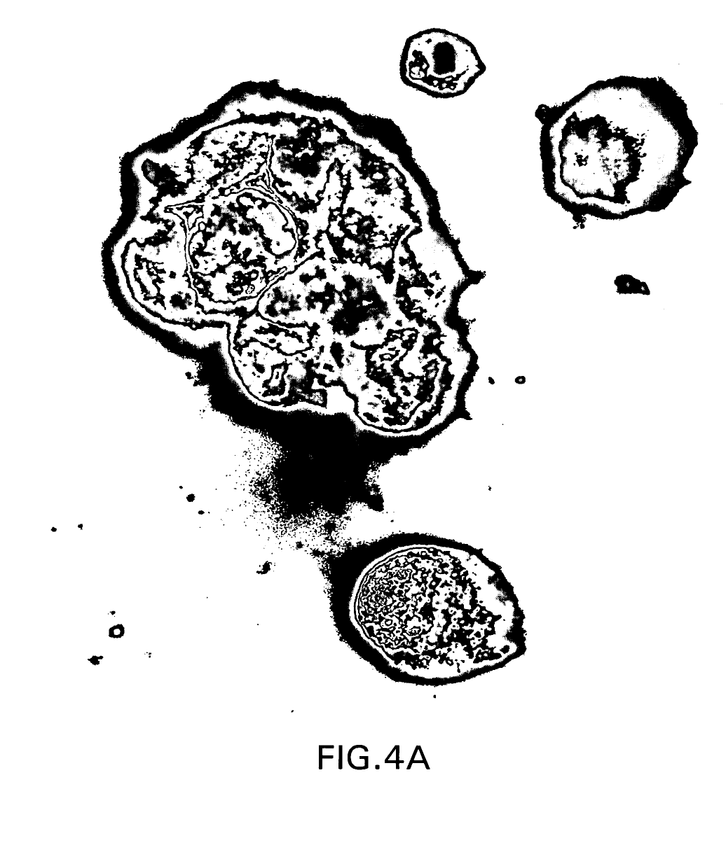

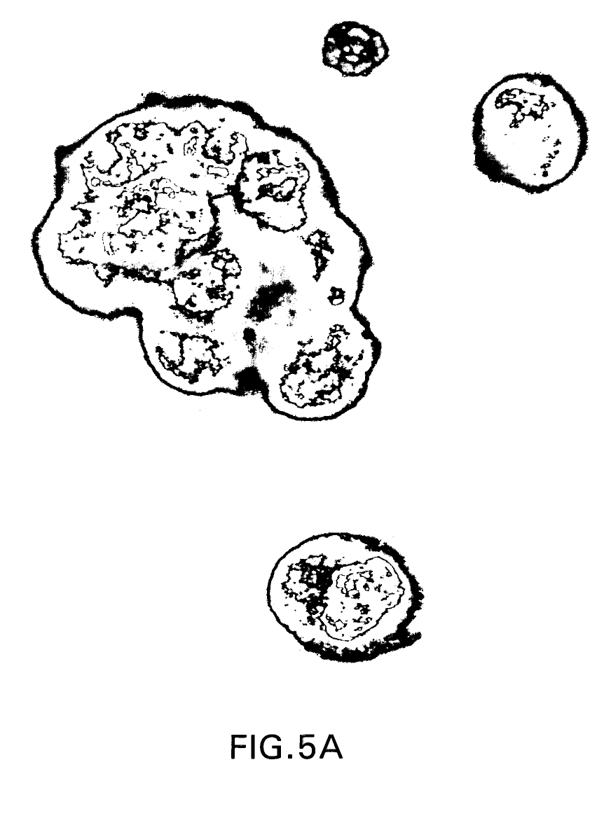

Multiple markers to identify cell type (e.g., cytokeratin), tissue-specific type (e.g., prostate specific marker antigen (PSMA), growth phase (PCNA and MiB1/Ki67) and cell growth inhibition (P27) have been used concurrently in the same cells and images showing successful staining are presented in FIG. 1. DAPI images have been used to determine DNA content for a measure of aneuploidy. LNCaP cells stained with anti-cytokeratin-FITC identify epithelial cells and with Ki67-CY3, which can be seen in some, but not all of the nuclei denote proliferating cells.

FIG. 1 shows five monochrome images of the same identical field of LNCaP cells obtained with five different filter cubes that can selectively distinquish DAPI, FITC, CY3, Texas Red, and CY5. The cells were incubated concurrently with four monoclonal antibodies, each conjugated to one of the above fluorophores, and then countered stained with DAPI. FIG. 1A) Image of cell nuclei stained with DAPI, a dye specific for DNA, obtained using a filter cube with a 360/40 nm exciter, a 400 nm dichroic and a 470/40 nm emitter. FIG. 1B) Image showing cellular cytokeratin stained with a monoclonal antibody-FITC conjugate and obtained using a filter cube with a 470/40 nm exciter, a 497 nm dichroic and a 522/40 emitter. FIG. 1C) Image showing the nuclear antigen, Ki67, stained with a monoclonal antibody-CY3 conjugate and obtained using a filter cube with a 546/11 nm exciter, a 557 nm dichroic and a 567/15 nm emitter. FIG. 1D) Image showing a prostate tissue marker, prostate specfic membrane antigen, stained with a monoclonal antibody-Texas Red conjugate and obtained using a filter with a 581/10 nm exciter, a 593 nm dichroic and a 617/40 nm emitter. FIG. 1E) Image showing the nuclear antigen, P27, stained with a monoclonal antibody-CY5 conjugate and obtained using a filter cube with a 630/20 nm exciter, a 649 nm dichroic and a 667/30 nm emitter.

Pseudocolor composite images of the five monochrome images (from FIG. 1) are available, but not included: A) Composite image showing cell nuclei stained with DAPI, blue, cytokeratin stained with FITC, green, and Ki67 nuclear antigen stained with CY3, red. B) Composite image showing cell nuclei stained with DAPI, blue, and prostate specfic membrane antigen stained with Texas Red, red. C) Composite image showing cytokeratin stained with FITC, green, and P27 nuclear antigen stained with CY5, red.

### EXAMPLE 2

Example 2 illustrates nuclear antigen stainging for growth markers and a growth inhibitor. Table 1 shows the results of several experiments and can be summarized as follows: confluent IRM90 cells, 50% of the nuclei are labeled with P27 and about 16% are labeled with MiB1; with exponential IRM90 cells, about 50% of the nuclei are labeled with MiB1 while none are labeled with P27. Using LNCaP cells, about 10% of the nuclei are labeled with PCNA (S-phase) while 50% are labeled with MiB1 and none are labeled with P27. Thus, in one assay, cells can be identified that are proliferating or in quiescent state, and it can also be determined if these same cells are neoplastic.

TABLE 1
Prostate Cancer Cell Lines and Fibroblast Cells
Nuclear Antigen Staining for Growth (MiB1 and
PCNA) or Growth Inhibition Factor (P27)

| Nuclear Antigen | | PCNA % | | |
|---|---|---|---|---|
| Cell | Growth | Labelled | MiB1% Labelled | P27% Labelled |
| Line | Status | Nuclei | Nuclei | Nuclei |
| IRM90 | Confluent | NA | 16% | 50% |
| IRM90 | Exponential | NA | 50% | 0 |
| LNCaP | Growing | 10% | 50% | 0 |

These preliminary results show that MiB1 is a better indicator of growth in this experiment than PCNA.

### EXAMPLE 3

This example illustrates the measurement of DNA Quantification Content. Quantifying the nuclear DNA content in single cancer cells in comparison to white blood cells can be used as a measure of aneuploidy. The fluorochrome, 4′,6-diamidino-2-phenylindole (DAPI), binds to DNA with high specificity and the complex exhibits intense fluorescence. This has permitted the measurement of DNA in nuclei, and viral particles (Rao, J. Y. et al, Cancer Epidemiology, Biomarkers & Prevention, 7: 1027-1033 (1998), and in breast cancer cells (Coleman, A W, et al, J. Histochem & Cytochem. 29: 959-968 (1981). The basis for the quantitative fluorescence image assay is a comparison of the DNA content with a reference cell, such as white blood cells (WBC) from the patient on the same slide with the circulating epithelial cell (CEC) in question. Circulating WBC are in the Go phase of the cell cycle have 2 copies (2c) of DNA (=2N) content. Normal epithelial cells in G₀ to G₁ phase also have 2c DNA and at G₂-M phase have 4c DNA. Therefore, a ratio of the reference WBC DNA content to CEC DNA content substantially greater than one is a specific measure of aneuploidy since a dividing cell with 3c or 4c DNA will have a 6c to 8c DNA content at G₂-M. The assay is completely controlled internally since the nuclear DAPI fluorescence of the WBC and the cancer cell are compared only on same slide and measured within very close proximity on the slide. This eliminates any problems that may arise from staining, e.g., incubation time or DAPI concentration, or from image acquisition or image processing since the reference and test cells are always treated exactly alike. Two prostate cancer cell lines (LNCaP & TSU) and normal prostate cells (NPC) were spiked into blood and the samples were processed using standard protocols for cell isolation and cell staining (U.S. Pat. No. 5,962,237). Larger numbers of LNCaP and TSU, as well as a third prostate cancer cell line (PC3) were spiked into isolated WBC and stained as above. Mounting medium contained DAPI (XHM003) at 0.5 ug/ml by diluting the normal stock 1:3. Fluorescence images of DAPI-stained nuclei were acquired using exposure times of 0.5 to 3 seconds. Background images were acquired with a slide that contained DAPI mounting medium but no cells. Prostate cells were identified by positive cytokeratin staining showing the presence of lableling.

DAPI fluorescence of WBC was linear with respect to exposure times of 0.5 to 3 seconds (for image acquisition) and DAPI concentration (0.5 to 1.5 ug/ml). The fluorescence per pixel should be below 2000 units per pixel to ensure linearity. For the blood-spiked samples, the ratio of LNCaP nuclear DAPI fluorescence to WBC DAPI fluorescence ranged from 1.9 to 4.4 (16 cells) indicating that the cells in this cancer cell line were essentially all aneuploid (greater than 2N DNA). For TSU cells the ratio ranged from 1.6 to 3.4 (13 cells) indicating that most (10 out of 13) had more than 2N DNA and therefore aneuploid. These results are supported by previous FISH data, which showed that these two prostate cancer cell lines are aneuploid with respect to chromosome 18. For NPC, cultured in the presence of mitogens, the NPC/WBC nuclear fluorescence ratios with respect to DAPI ranged from 1.0 to 1.5. Data from anti-Ki67-treated cells show that greater than 80% of NPC, grown in the presence of FBS, are in the growth phase of the cell cycle and should have NPC/WBC ratios greater than one. When larger numbers of cancer cells were spiked into isolated WBC, cytospun onto slides, and then analyzed to obtain the integrated fluorescence intensity of nuclear-bound DAPI, the data are as follows: LNCaP, 128 WBC & 56 cancer cells analyzed, 95% had greater than 2N content of DNA; TSU, 89 WBC & 125 cancer cells analyzed, 90% had greater than 2N content of DNA; PC3, 95 WBC & 90 cancer cells analyzed, 94% had greater than 2N content of DNA.

The human karyotype is very tight, therefore aneuploidy is an excellent marker for identifying cancer cells. Any CEC whose CEC/WBC nuclear DAPI fluorescence ratio is greater than two (more than 4N content of DNA) should be considered neoplastic (see LNCaP model). Over 95% of the cells in normal differentiated prostate tissue should be in G₀/G₁ phase of the cell cycle (=2N DNA). Therefore, the finding of any CEC of prostate origin (positive staining staining for WDZ) in the peripheral blood should be suspect, especially if the cell has a CEC/WBC nuclear DAPI fluorescence ratio of 1.3 or greater. Such cells could be aneuploid since the majority of normal prostate cells would not have greater than 2N content of DNA, viz., a CEC/WBC of approximately one).

TABLE 2
WBC versus Normal Prostate Cells (NPC)

| | | WBC | | NPC | |
|---|---|---|---|---|---|
| Image | Cells # | IFI × 1000 | Range | IFI | npc/wbc |
| 2 | 9 | 234 | 196-271 | 267 | 1.14 |
| 3 | 11 | 274 | 209-344 | 344 | 1.26 |
| 4 | 11 | 328 | 282-377 | 478 | 1.46 |
| 6 | 9 | 270 | 209-317 | 363 | 1.34 |
| 8 | 11 | 218 | 184-233 | 346 | 1.59 |
| | | | | 298 | 1.37 |
| 10 | 12 | 268 | 213-330 | 419 | 1.56 |
| 11 | 10 | 324 | 297-353 | 313 | 0.97 |
| 12 | 10 | 275 | 234-304 | 266 | 0.97 |

*Integrated Fluorescence Intensity (IFI = area in pixels × fluorescence/pixel)
Average WBC IFI for eight different images from the same slide is 274000 with a standard deviation of 38000. Average WBC area, in pixels, for the eight different images ranged form 729 to 1019. Area of NPC ranged from 1159 pixels to 1651 pixels.

### EXAMPLE 4

This example illustrates staining of cells for androgen receptor detection.

The method for immunohistochemical staining of androgen receptor in circulating cancer cells from cancer patients is outlined below: Obtained about 20 ml of blood from cancer patients diagnosed with prostate cancer; Blood was processed by double gradient centrifugation system and interfaces were collected into new tubes; leukocytes in the interface suspension were depleted by magnetic cell sorting system; The cells from magnetic cell sorting system were spun on the slides through cytospin; The slides were fixed in 2% paraformaldehyde; Slides were washed 3 times for 2 minutes in PBS and incubated with blocking serum in PBS-gelatin for 20 min.; Androgen Receptor antibodies: 1). Mouse IgG against human androgen receptor a.a.1-21 (a gift for Dr. Gail Prins of the University of Illinois at Chicago), 2). Mouse IgG against human androgen receptor a.a. 33-485 (PharmMingen), 3). Mouse IgG against human androgen receptor 486-651 (PharmMingen). Androgen receptor antibody dye conjugation: 1). Direct dye conjugation: TEXAS RED™, CY3, TRITC and FITC.

Indirect immunohistochemistry staining: Different dye conjugated anti mouse IgG antibody (e.g., secondary antibodies: Rhodamine labeled Goat anti-Mouse IgM, Fluorescein labeled Goat anti-Mouse IgG (H+L), Anti-Mouse IgG (H+L), F(ab′)2-FITC (Goat), TEXAS RED™-X Goat anti-Mouse IgG (H+L).

Immunohistochemistry staining: The slides were incubated with 1^{st} antibody at RT for 60 minutes. The slides were incubated with 2^{nd} antibody-dye at RT for 60 minutes. DAPI counterstained for 10 minutes. Examined under microscope.

Detection of androgen receptor gene copy number is discussed below.

Fluorescent in situ hybridization (FISH) with gene-and locus-specific probes provides a rapid means to assess copy numbers of specific sequences in individual interphase nuclei. Recent technical improvements have made FISH applicable to the analysis of both fresh and archival tissue specimens in research as well as in diagnostic laboratories. FISH is limited to analysis of one or a few loci at a time, making genome-wide surveys impractical. The use of this technique will be illustrated in the analysis of genetic changes in circulating cancer cells. The probes which have been used for in situ hybridization are either LSI androgen receptor genomic DNA from the locus of Xq12 (Vysis Inc.) or the PCR products which are generated by a specific androgen receptor gene primers with genomic DNA as a template and labeled by nick translation kit (Vysis Inc.) containing Spectrum Orange dUTP.

Fluorescent In Situ Hybridization (FISH): FISH Cocktail (Vol./slide): 17.0 μl FISH buffer; 1.0 μl Xq12 probe-Spectrum Orange (Vysis; Lot#13156); 2.0 μl H₂O. FISH Staining: Add the FISH cocktail onto the sample area on the slide; Place the coverslip on the sample area; Seal the coverslip with rubber cement; Denature the sample at 85° C. for five minutes on a hot plate; Hybridize the sample at 42° C. in oven for four hours in a moisture box; Take off the rubber cement and coverslip form the sample slide very carefully; Wash the slide in a Coplin Jar with 2×SSC/0.1% NP-40 (USB; Cat: 19628) at 52° C. (preheated) for 2 minutes; Air-dry the slide at RT; Counterstain the sample with 14 μl/slide of DAPI in mounting medium (1.0 μg/ml; Vector Lab; Cat. H-1200); Place a coverslip on the sample; Seal the coverslip with FLO-TEXX mounting medium (Lerner Lab; Cat. M770-3); Stand the slide in a dark area at RT for at least 10 minutes; and analyze the stained slide under fluorescent microscope.

TABLE 3
Percentage of LNCaP cells with Androgen
Receptor Gene Copy Number

| 2 Copies | 3 Copies | 4 Copies | 5 or more Copies |
|---|---|---|---|
| 75% | 15% | 8% | 2% |

TABLE 4
Androgen Receptor Gene Copy Number in Circulating
Cancer Cells from Cancer Patients

| Patient's No. | 1 Copy | 2 Copies | | 3 Copies | 4 Copies |
|---|---|---|---|---|---|
| #80150 | 1 cell | | | | |
| #80154 | 1 cell | 1 | cell | 1 cell | |
| #80189 | | 3 | cells | 1 cell | 1 cell |
| #80199 | 2 cells | 34 | cells | 7 cells | 4 cells |

All of the references cited herein, including patents and patent applications, are hereby incorporated in their entireties by reference.

While the invention has been described and disclosed herein in connection with certain preferred embodiments and procedures, it is not intended to limit the invention to those specific embodiments. Rather it is intended to cover all such alternative embodiments and modifications as fall within the spirit and scope of the invention.

1-58. (canceled)

59. A method of characterizing single circulating epithelial cancer cells, other than prostate cancer cells, obtained from about 5 mL to 75 mL of blood comprising:
concurrently measuring multiple cellular markers using fluorescent probes, wherein said probes emit different wavelengths of light to distinguish multiple cellular markers expressed in said cells using fluorescence microscopy.


SEQUENCE LISTING (12 sequences)

SEQ ID NO: 1 - DNA, 54 bp, Artificial Sequence
  Probe
ttttgagatgtgtgtactcacactaagagaattgaaccaccgttttgaaggagc

SEQ ID NO: 2 - DNA, 54 bp, Artificial Sequence
  Probe
tttttgtttcaaacgtgaactttgaaaggaaagttcaactcggggatttgaatg

SEQ ID NO: 3 - DNA, 54 bp, Artificial Sequence
  Probe
ttttgctgtggcattttcaggtggagatttcaagcgatttgaggacaattgcag

SEQ ID NO: 4 - DNA, 54 bp, Artificial Sequence
  Probe
ttttggatttggcggctggaggaggtcacatctctggatgactgcgatccagag

SEQ ID NO: 5 - DNA, 54 bp, Artificial Sequence
  Probe
ttttatcttggcgagatcggtgcccggagcggaatccacctccacactgacctg

SEQ ID NO: 6 - DNA, 54 bp, Artificial Sequence
  Probe
ttttttgaactgtgtctccacgtcgtggacattgatggtaccttctcggaaggc

SEQ ID NO: 7 - DNA, 57 bp, Artificial Sequence
  Probe
ttttgtgcagaccgtgtccccgcagggcagaaggctgctcagaaaccggcgggccac

SEQ ID NO: 8 - DNA, 20 bp, Artificial Sequence
  Probe
cgatttgaggacaattgcag

SEQ ID NO: 9 - DNA, 30 bp, Artificial Sequence
  Probe
gtactcacactaagagaattgaaccaccgt

SEQ ID NO: 10 - DNA, 21 bp, Artificial Sequence
  Probe
gacgatggagtttaactcagg

SEQ ID NO: 11 - DNA, 41 bp, Artificial Sequence
  Probe
tcgttggaaacgggaataattcccataactaaacacaaaca

SEQ ID NO: 12 - DNA, 29 bp, Artificial Sequence
  Probe
aagccttttcctttatcttcacagaaaga